(19) United States
(12) Patent Application Publication
(10) Pub. No.: US 2008/0287813 A1
(43) Pub. Date: Nov. 20, 2008

(54) Blood Pressure Monitoring Device and Methods for Making and for Using Such a Device

(75) Inventors: Kay-Uwe Kirstein, Dietikon (CH); Tomi Salo, Zurich (CH); Jurg Grunenfelder, Zurich (CH); Jan Sedivy, Zurich (CH); Gregor Zund, Herrliberg (CH); Oliver Brand, Atlanta, GA (US)

Correspondence Address: MARK D. SARALINO (GENERAL);RENNER, OTTO, BOISSELLE & SKLAR, LLP, 1621 EUCLID AVENUE, NINETEENTH FLOOR, CLEVELAND, OH, 44115-2191, US

(73) Assignees: EIDGENOSSISCHE TECHNISCHE HOCHSCHULE ZURICH, Zurich (CH); UNIVERSITAT ZURICH, Zurich (CH)

(21) Appl. No.: 10/599,187

(22) PCT Filed: Mar. 3, 2004
(86) PCT No.: PCT/IB2004/000953

§ 371 Date: Jul. 30, 2008

(51) Int. Cl. A61B 5/02; A61B 5/04

(52) U.S. Cl. 600/488; 600/485; 600/395; 600/372

(57) ABSTRACT

Disclosed are a system and a method for noninvasively and continuously monitoring blood pressure. Also disclosed is a method for making such a device. The system includes a semiconductor chip comprising a transducer array of individual pressure or force sensors and associated circuitry providing control signals to and/or processing signals from these sensors, all of the above integrated in the chip. Also disclosed is a specific sensor structure provided on said chip. The invention further encompasses a system for measuring and/or tracking the blood pressure waveform and for combining the latter with related blood values like the heartbeat, derived from the above or other measuring devices.

### TECHNICAL FIELD

The present invention relates to a device and a method for noninvasively monitoring blood pressure. The apparatus includes a semiconductor chip comprising a transducer array of individual pressure or force sensors and associated circuitry providing control signals to and/or processing signals from these sensors, integrated in the chip. Also disclosed is a specific sensor structure provided on said chip. The invention also encompasses a system for measuring and/or tracking the blood pressure waveform and combining the latter with related blood values like the heartbeat, derived from the above or other measuring devices.

### BACKGROUND AND PRIOR ART

Measurement of blood pressure is one of the most common procedures done during examination of a patient in hospitals. It is usually done with the aid of a cuff attached to the arm, which only gives an indication of two values, namely the systolic and the diastolic pressure. Especially during surgery and treatment at the intensive care unit, a continuous measurement of the blood pressure is required. This is routinely done using an intra-vascular catheter, where the blood pressure is compared to a pressure of the liquid inside the catheter tubing. Since this is an invasive method, it is used only when it is absolutely necessary. However, in many cases a continuous measurement would be beneficial for the medical personnel in the evaluation of the patient's condition. Furthermore, inserting a catheter into a small child or severely ill person with very weak blood vessels is extremely difficult, even impossible. Thus there exists a need for simple extra-vascular method for measuring the blood pressure giving continuous signal.

The continuous measurement of blood pressure by use of arterial tonometer transducers is known in the art as disclosed in two Eckerle U.S. Pat. Nos. 4,269,193, 4,802,488, for example.

Eckerle U.S. Pat. No. 4,802,488, cited above, discloses how intraarterial blood pressure can be measured noninvasively by an electromechanical transducer that includes an array of transducer elements. The transducer extends across an artery with the transducer elements extending across the artery. Diastolic and/or systolic pressure and pulse amplitude values are obtained from the outputs of the transducer elements, which values are stored in computer. Information concerning the subject, i.e. the patient, related to the diameter of the underlying artery including, for example, the subject's age, weight, arm and wrist diameter may also be entered into the computer, from which information an estimation of the diameter of the underlying artery is obtained.

Using the set of pulse amplitude values, the particular transducer element or elements located substantially at the center of the measurement area is/are identified and the outputs from only said particular transducer element(s) used for monitoring the subject's blood pressure and/or for further processing.

The device and method according to Eckerle U.S. Pat. No. 4,802,488 above appears to be workable and is probably implemented in the device described in the above-cited internet publication. However, looking at the device shown in said publication “Verfahren der Arteriellen Tonometrie”, it becomes clear that the use of this cuff-like, bracelet-type device is limited to so-to-speak normal applications, i.e. applications where there is sufficient space at and around the measurement area and where sterility is of no great concern. In an operating room or even within a human body during surgery, it is hardly conceivable how the described prior art device may be used.

An internet publication, http://www.dr-kaiser-medizintechnik.de/blutdruck.htm, shows a blood pressure measuring device that appears to incorporate at least part of the technology disclosed in the two U.S. patents cited above. The Colin BP-508 T CS device shown there is of the bracelet-type, looking rather robust, but consequently being of substantial size and requiring a specific position of the patient.

A similar device is disclosed by L. A. Steiner et al in the journal “Anaesthesia”, 2003, vol. 58, pages 448-454, entitled “Validation of a tonometric noninvasive arterial blood pressure monitor in the intensive care setting”. The CBM-700 shown and described therein is again a rather large hand-cuff device that is to be attached to the wrist or arm of the patient.

S. Terry, J. S. Eckerle et al disclose in “Silicon Pressure Transducer Arrays for Blood-pressure Measurement”, in “Sensors and Actuators”, A21-A23 (1990), pages 1070-1079, a tonometer transducer array in which several transducers share a common diaphragm. The device is fabricated from silicon using anisotropic etching and includes piezoresistors for signal generation. However, no other electrical or other elements are provided on or in the silicon body.

The present invention leads to a new approach, providing a remedy to many disadvantages of prior art devices. By reducing size and power consumption of a transducer device significantly, a wide spectrum of new applications is accessible, e.g. intra-body uses during surgery. By shaping the sensor array on such a transducer accordingly, one can improve and simplify blood pressure signal reception and evaluation. By speeding up signal processing, critical situations may be detected early enough to avoid problems in a time-critical environment, e.g. during surgery or after a heart attack.

Also, reducing power consumption and processing the sensor signals “on chip” may even—when a small power source is included on the chip—allow wireless data transmission and thus provide for a fully independent device for monitoring the blood pressure. Needless to say that this opens a variety of further applications akin to today's widespread use of cardiac pacemakers.

### BRIEF SUMMARY OF THE INVENTION

This invention is based on detecting the continuous force signal generated by a blood vessel. The origin of this force is the overpressure contained inside the vascular system. One or more force measurement instruments may be placed extravascularily, such as on the skin or the heart surface.

The force variations are recorded continuously, whereby the continuous blood pressure is extracted from these force variations. The thus derived data can be further used to extract the relative difference between systolic and diastolic pressure.

If it is necessary to obtain absolute values, often required for the evaluation of the patient's condition, high and low extremes of the force signal need to be calibrated. This may be done by using a separate measurement device, e.g. a usual handcuff blood pressure meter.

The present invention now creates a novel approach for such a non-invasive blood measuring device in that it integrates the electromechanical sensor and at least some of the associated circuitry onto a single chip.

This leads to a number of advantages, including:

The possibility of making of sensor and circuitry by essentially the same semiconductor manufacturing process, in particular a CMOS process, data transmission speed and reliability are improved and the error probability reduced. It may also results in lower production cost of the whole blood pressure measuring device.

The produced chip is much smaller and lighter and has a lower power consumption than prior art devices, which opens new possibilities of its use, in particular the use in antiseptic environments as an operation room or even within a human body during surgery.

By arranging a plurality of sensors in an array adapted to the particular use, even complex measurements can be executed, for example spatially and/or timely distributed measurements to determine the characteristics of blood flow, the “blood wave”, in a blood vessel.

Spatially and/or timely distributed measurements using plurality of sensors in an array allow also locating and identifying arteries and veins running underneath a tissue, i.e. myocardial tissue on a heart surface, based on directional information from an array and characteristic signal features of arteries and veins. Furthermore, abrupt features, such as blockages due to calcification inside arteries and veins, can be identified based on a map pattern of the blood pressure data.

These and other advantages and details will be apparent from the subsequent description of an embodiment of the invention.

### BRIEF DESCRIPTION OF THE DRAWINGS

The present invention and its advantages will be better understood when the written description provided herein is taken in conjunction with the drawings wherein:

FIG. 1 is a top view of a single transducer element;

FIG. 2 is a cross-sectional view along line A-A′ of the single transducer element in FIG. 1;

FIG. 3 is a top view of a two-by-two array of transducer elements;

FIG. 4 is a cross-sectional view along line B-B′ of the two-by-two transducer array in FIG. 3;

FIG. 5 is a layout of the monolithic integration of a two-by-two transducer array with electronic circuitry;

FIG. 6 is a block diagram of an integrated transducer chip;

FIG. 7 is a block diagram of a whole system for measuring and recording deformation of a blood vessel wall;

FIG. 8: shows the method of measuring the deformation of a blood vessel wall,

FIG. 9 is a top view of a single transducer element of a second embodiment;

FIG. 10 is a cross-sectional view along line C-C′ of the single transducer element in FIG. 9;

FIG. 11 is a cross-sectional view along line D-D′ of the single transducer element in FIG. 9;

FIG. 12 is a layout view of a Wheatstone bridge configuration in the crosslinked beam structure in FIGS. 9, 10 and 11; and

FIG. 13 is a top view of a two-by-two array of transducer elements of FIG. 9.

For the sake of clarity, the figures do not necessarily show the correct dimensions, nor are the relations between the dimensions always in a true scale.

### DESCRIPTION OF PREFERRED EMBODIMENTS

When describing the details of two embodiments of the present inventions, it should be clear that the following description is directed to persons having a thorough understanding of the technology involved. For background information please refer to the paper by H. Baltes and O. Brand: “CMOS-based Microsensors”, in Sensors and Actuators A 92 (2001), pages 1-9 and to the book “VLSI Technology”, ed. by S. M. Sze, McGraw Hill, New York, 1988, which are both incorporated herein by reference.

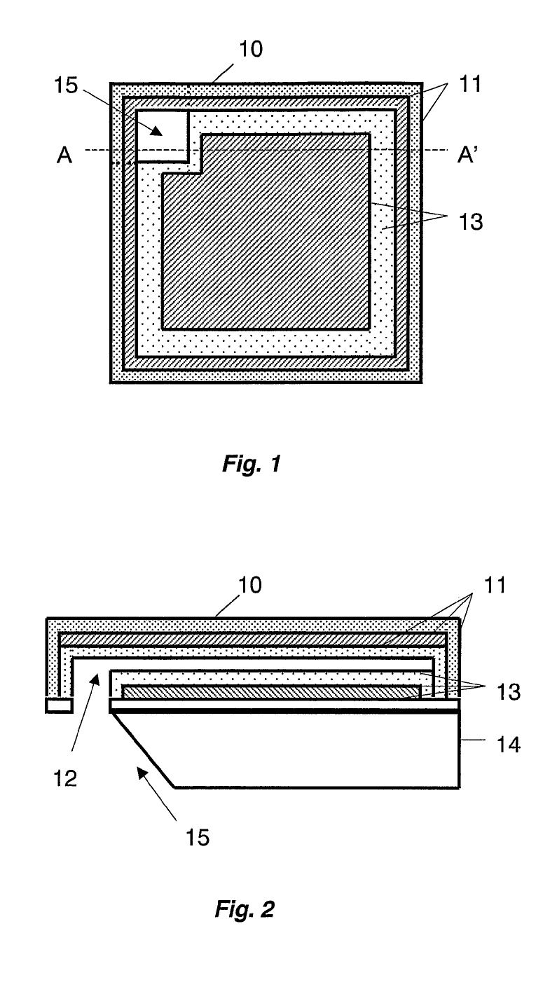

FIGS. 1 and 2 show the structure of an individual transducer element according to the present invention, whereby FIG. 2 is a cross section of FIG. 1.

The individual transducer element 10 includes several parts. An elastic membrane 11 with a side length of less than 150 μm consists of a top electrode layer with support and protection layers. The support and protection layers of the elastic membrane 11 are made using standard CMOS techniques, e.g. deposited silicon dioxide and oxynitride. The top electrode is a CMOS metal layer, which is deposited aluminum in this embodiment. The thickness of the membrane is about 3 μm. A fluid gap 12 allows the membrane 11 to deflect, the height of the fluid gap 12 being less than 1 μm. This fluid gap 12 is made by etching a material layer or layers through inherent structural layers, i.e. substrate 14 and bottom electrode 13 support layer. In this embodiment, the material layer that is removed to form the fluid gap 12 is deposited aluminium. Rigid bottom electrode 13 has an electrode layer with support and protection layers. These support and protection layers of the bottom electrode 13 are also made using standard CMOS techniques, e.g. deposited silicon dioxide and polysilicon, and thermally oxidized silicon dioxide. The parts 11, 12 and 13 are built onto a substrate 14 whose thickness is some hundreds μm. In order to allow for the sacrificial release of the elastic membrane 11 by creating the fluid gap 12, an opening or several openings 15 are etched through the substrate 14.

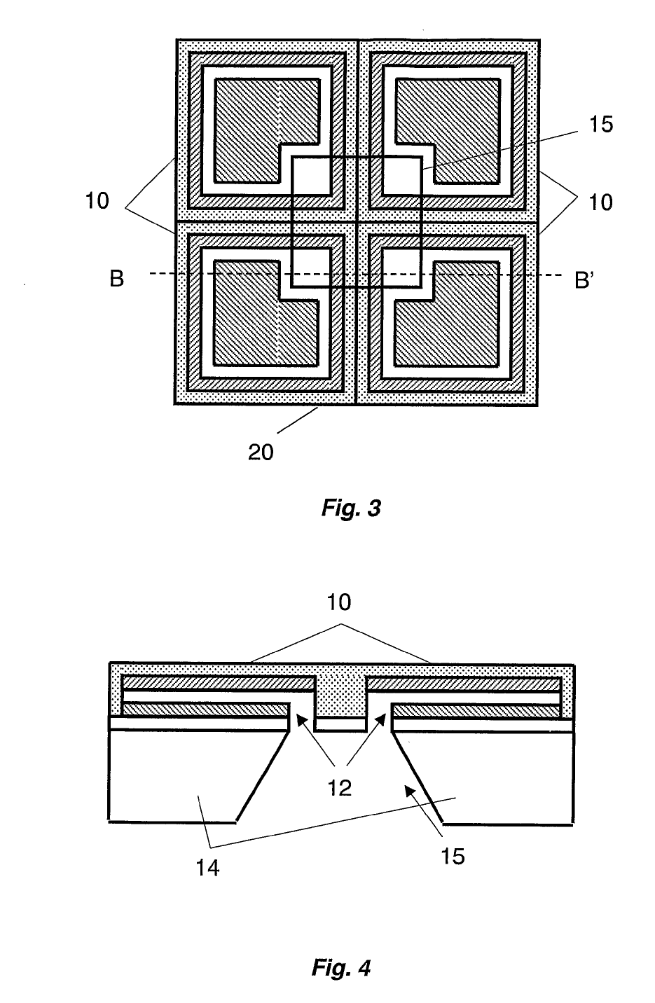

FIGS. 3 and 4 show a two-by-two array of four transducer elements, whereby FIG. 4 is a cross section of FIG. 3 along B-B′.

An array 20 is formed of single transducer elements 10. In this particular embodiment, the array 20 is made of two rows and two columns of the single transducer elements 10. The pitch of the neighbouring transducer elements 10 is less than 200 μm. In the embodiment shown with a single opening 15 through the substrate 14, this opening 15 is shared by four neighbouring transducer elements 10. The fluid gaps 12, cf. also FIG. 2, are then formed simultaneously to all transducer elements 10.

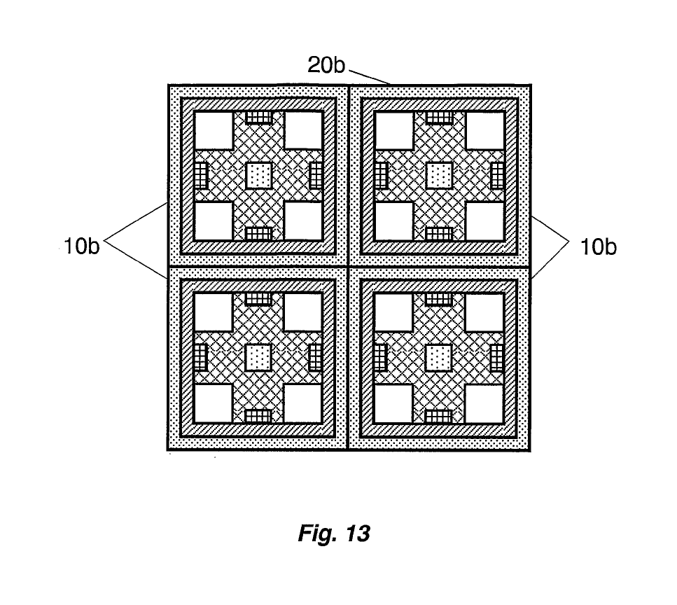

A second embodiment is shown in FIG. 13 and will be described in detail further down.

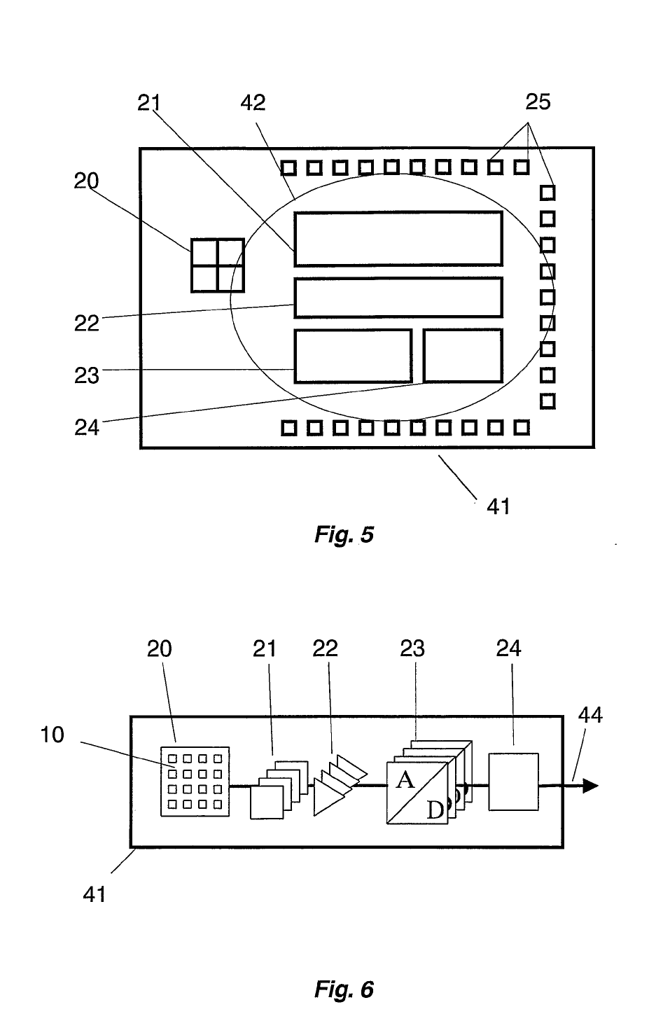

FIG. 5 is a layout of a monolithically integrated chip of a two-by-two transducer array together with integrated electronic circuitry according to the invention. In the embodiment shown, an array 20 of transducer elements 10 is monolithically integrated onto a single substrate 14 together with the signal readout system 42. In another embodiment, a transducer array 20b of transducer elements 10b is integrated.

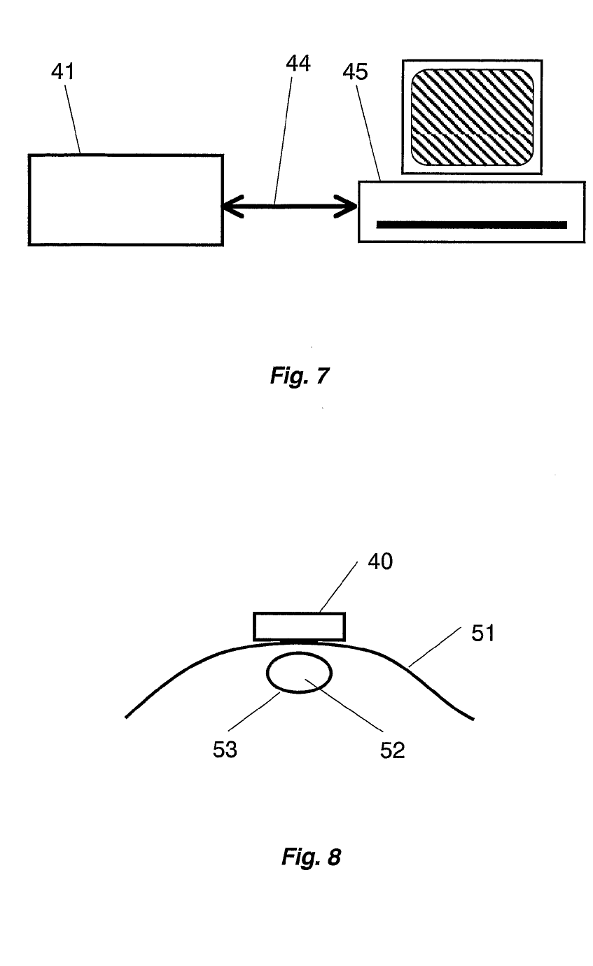

When using capacitive transducer elements as in this first embodiment, a typical signal readout system 42 consists of a readout circuit 21, a signal conditioning circuit 22, an analog-to-digital converter circuit 23 and an interface circuit 24. The transducer chip is connected to interfacing system 44 via contact pads 25. The interface 44, c.f. FIGS. 6 and 7, is in this embodiment an electrical cable. In other embodiments, this may be replaced by a wireless connection.

In the second embodiment of a transducer array 20b, described further down in FIG. 13, a typical signal readout system 42 consists of the same functional blocks.

FIG. 6 shows a block diagram of an embodiment of the integrated transducer chip, comprising a 4×4 array of single transducer elements 20, a readout circuit 21, a signal conditioning circuit 22, an analog to digital converter circuit 23 and an interface circuit 24. The transducers 10 are electrically connected to a readout circuit 21, which in turn is connected to a signal conditioning circuit 22. In the preferred embodiment, several transducer elements 10 share one readout circuit 21 and one signal conditioning circuit 22 through a multiplexing scheme, where each transducer element 10 is addressed individually.

The signal at the output of the signal conditioning circuit 22 is connected to analog to digital converter circuit 23. In other embodiments several signal conditioning circuits 22 and analog to digital converter circuits 23 may be used in parallel. In the preferred embodiment, the readout circuit 21, the signal conditioning circuit 22 and the analog to digital converter circuit 23 are realized as a sigma-delta modulator circuits with decimation filtering. An interface circuit 24 is connected to the output of said analog to digital converter circuit 23 to provide a connection to an external device via a said interface 44.

FIG. 7 shows a block diagram of an embodiment of a whole measurement and recording system. An interface 44 connects the integrated transducer chip 41 to a computer system 45 which evaluates the transmitted data and provides suitable outputs.

FIG. 8 finally shows a method of monitoring the blood pressure by measuring the deformation of a blood vessel wall. The sensing device 40 is an assembled structure consisting of the said integrated transducer chip 41, described in detail above, a base plate for mechanically holding this transducer chip 41, and some polymer layers for protection and biocompatibility, for example. The sensing device 40 is attached to the surface of an organ 51, such as the skin or the heart.

The sensing device 40 somewhat deforms the blood vessel 52 by deforming the surface of the organ 51 in order to sense the movement of the blood vessel wall 53 vertical to elastic membranes 11 of the transducer chip 41 in the sensing device 40. This movement deflects a membrane 11 of a transducer element 10. The distance between top electrode in membrane 11 and bottom electrode 13 changes in response to the deflection of the membrane 11. In this particular embodiment, the change in mutual distance of the electrodes changes the capacitance of the electrode system. Thus the displacement of the vessel wall 53 can be read out as a change in capacitance in transducer element 10.

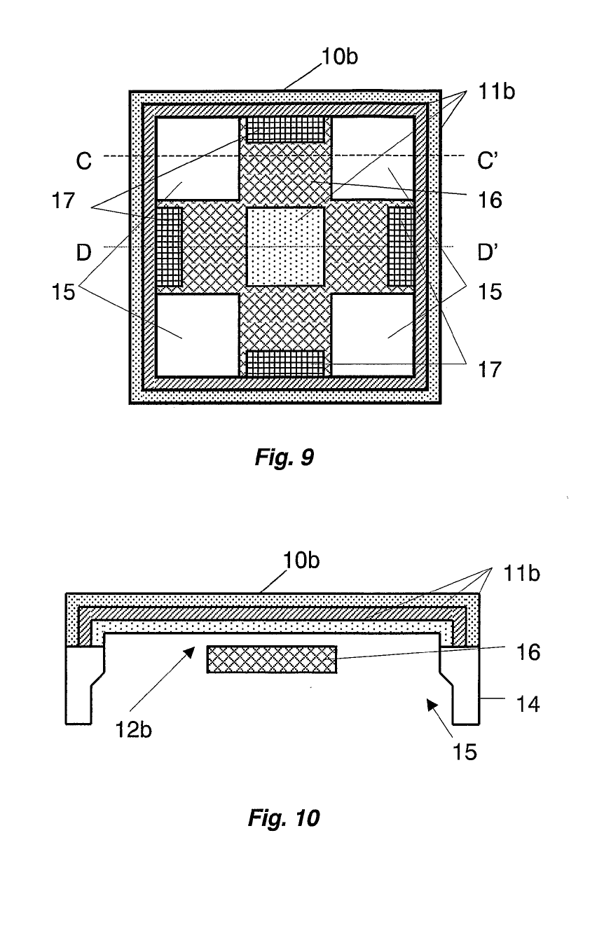

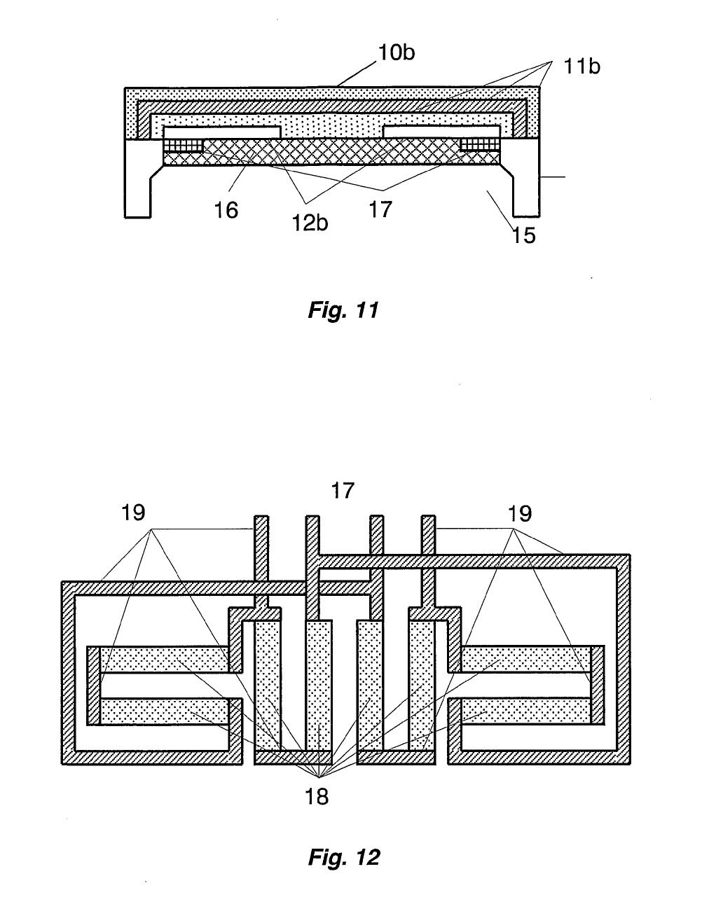

FIGS. 9, 10 and 11 show the structure of a second embodiment of an individual transducer element according to the present invention, whereby FIGS. 10 and 11 are cross sections of FIG. 9.

In this second embodiment, the individual transducer element 10b includes several parts. A membrane 11b, having a side length of less than 250 μm, is suspended over a cross-linked beam structure 16 and connected to it at the center. The membrane 11b provides mechanical and electrical protection and is made of standard CMOS deposited silicon dioxide, metal (in this embodiment aluminum), and oxynitride. The membrane 11b is about 3 μm thick. The cross-linked beam structure 16 is formed using an implanted n-well of a standard CMOS process. The cross-linked beam structure 16 has a thickness of about 6 μm.

Close to the support point of each beam in said beam structure 16 are resistors 18 connected with conductor lines 19 to a Wheatstone bridge configuration 17, shown in FIG. 12 in detail. The resistors 18 are made by a standard CMOS p-doping process and the conductor lines are CMOS metal, in this embodiment deposited aluminum. A fluid gap 12b decouples the cross-linked beam structure 16 from the membrane 11b except at the center. The height of the fluid gap 12b is less than 1 μm; it is manufactured by etching a material layer or layers through inherent structural layers, i.e. substrate 14 and crosslinked beam structure 16. In this embodiment, the material layer which is removed to form the fluid gap 12b, is deposited aluminum. The parts 11b, 12b and 16 are built onto a substrate 14 whose thickness is some hundred pm. To allow for the sacrificial release of elastic membrane 11b by creating the fluid gap 12b, several openings 15 are etched through the substrate 14 and the cross-linked beam structure 16.

FIG. 13 shows a two-by-two array of four transducer elements 10b. In this second embodiment, an array 20b is formed of two rows and two columns of the single transducer elements 10b. The pitch of the neighbouring transducer elements 10b is less than 300 μm.

In this embodiment, the movement of the blood vessel wall 53 deflects the connected system of a membrane 11b and a cross-linked beam structure 16 in transducer element 10b. The deflection of said cross-linked beam structure 16 changes the electric resistance of the resistors 18 connected into a Wheatstone-bridge configuration 17 with conductor lines 19. The change in one or several resistors 18 in the Wheatstone-bridge configuration 17 changes the electric voltage output of said Wheatstone bridge. Thus the displacement of the blood vessel wall 53 can be read out as a change in the output voltage of the Wheatstone-bridge 17 in transducer element 10b.

In other embodiments, the vertical movement of the blood vessel wall 53 may effect the change other electrical values like inductance or voltage.

Through an electrical connection of the transducer element 10 to the readout circuit 21, the change in the electrical measure, capacitance in the first embodiment, is converted to an electric voltage signal. In other embodiments, the signal may be delivered as electric current. In the second embodiment, the transducer element 10b provides an electric voltage signal through a readout circuit, as shown in FIGS. 5 and 6, embedded in said transducer element 10b. A connected signal conditioning circuit 22 performs filtering and amplification of said electric voltage signal from said readout circuit 21 and a analog-to-digital converter 23 provides the amplified and filtered data to the interface circuit 24 in digital format. In the preferred embodiment, the interface circuit 24 delivers the data to the interface 44 via contact pads 25.

The computer 45, see FIG. 7, receives said data via said interface system 44 and records it as continuous blood pressure data. The computer may also calculate the systolic, diastolic and mean blood pressures and/or the heart stroke volume from the recorded continuous blood pressure data. When directional information is processed from the continuous blood pressure data, it can be used to locate arteries and veins running underneath a tissue, i.e. myocardial tissue on heart surface. Based on the characteristic blood pressure features of arteries and veins, closely together running blood vessels can be identified. Furthermore, abrupt features, such as blockages due to calcification inside arteries and veins, can be identified based on a map pattern of the recorded continuous blood pressure data.

While the present invention has been described by way of a few examples, these shall not limit the scope of protection since it is obvious to someone skilled in the art that the invention can be easily adapted to match many requirements in the field of blood pressure measuring transducers and systems, including their design and/or manufacturing and integration.

1. A transducer for continuously and noninvasively measuring blood pressure in a blood vessel, having integrated into a single semiconductor chip:
an array of transducer or sensor elements,
means for reading out data from said sensor elements, and
means for converting said data into a transmittable form.

2. The blood pressure transducer according to claim 1, further including interface means for transmitting said data from said chip to an external computer.

3. The blood pressure transducer according to claim 1, wherein the semiconductor chip is a CMOS chip.

4. The blood pressure transducer according to claim 1, wherein each sensor element comprises a fluid-filled capacitive sensor having a flexible electrode or membrane and a rigid electrode and a fluid gap connected to an opening.

5. The blood pressure transducer according to claim 1, wherein each sensor element comprises a resistive sensor having strain sensitive resistors on a flexible structure of cross-linked beams a flexible protective membrane and a fluid gap connected to openings.

6. The blood pressure transducer according to claim 1, wherein the sensor elements are arranged in a square array.

7. The blood pressure transducer according to claim 6, wherein the array comprises at least 2×2 sensor elements, preferably 4×4 sensor elements.

8. The blood pressure transducer according to claim 4, wherein the array comprises 2×2 sensor elements arranged with adjacent openings located in the center of said array.

9. The blood pressure transducer according to claim 7, wherein the array of sensor elements is placed close to one end of the semiconductor chip.

10. The blood pressure transducer according to claim 1, wherein the semiconductor chip is part of a sensing device which further includes a power source.

11. The blood pressure transducer according to claim 2, wherein the interface means for transmitting the data from said chip to an external computer is a wireless transmission means.

12. A system for continuously and noninvasively measuring and monitoring blood pressure in a blood vessel, comprising a sensing device including a single semiconductor chip having integrated:
an array of sensor elements overlying said blood vessel,
means for reading out data from said sensor elements,
means for converting said data, and
means for interfacing with external evaluation means.

13. The measuring and monitoring system according to claim 12, further including a power source on the sensing device and wireless means for interfacing with the external evaluation means.

14. A method for making a transducer for continuously and noninvasively measuring and monitoring blood pressure in a blood vessel, the method including the steps of:
fabricating on a single semiconductor chip with conventional semiconductor technology, preferably CMOS technology,
an array of sensor elements,
means for converting, and
means for transmitting said data to external evaluation means.

15. A method for using a transducer according to claim 1 for continuously and noninvasively measuring and monitoring blood pressure in a blood vessel, the method comprising the steps of:
extracting directional information from said continuous blood pressure measurement data to locate arteries and/or veins, and/or
extracting characteristic signal features from said continuous blood pressure measurement data to differentiate between arteries and veins.

16. A method for using a transducer according to 11 claim 1 for continuously and noninvasively measuring and monitoring blood pressure in a blood vessel, the method comprising the steps of:
producing a map pattern of said continuous blood pressure measurement data to identify abrupt features, in particular blockages due to calcification inside arteries and veins.
